(19) United States
(12) Patent Application Publication
(10) Pub. No.: US 2012/0253075 A1
(43) Pub. Date: Oct. 4, 2012

(54) METHOD FOR PRODUCING CARBON NANOMATERIALS AND/OR CARBON MICROMATERIALS AND CORRESPONDING MATERIAL

(75) Inventors: Jens Helbig, Nuernberg (DE); Christian Zenkel, Gesees (DE)

(73) Assignee: FUTURECARBON GMBH, Bayreuth (DE)

(21) Appl. No.: 13/496,983

(22) PCT Filed: Sep. 16, 2010
(86) PCT No.: PCT/EP2010/063642

§ 371 Date: Jun. 8, 2012

(30) Foreign Application Priority Data
Sep. 17, 2009 (DE) 10 2009 041 944.6

(51) Int. Cl. C07C 211/54; C07C 25/22; C07C 17/269; B82Y 40/00; B82Y 30/00

(52) U.S. Cl. 564/426; 570/183; 570/190; 977/748; 977/847

(57) ABSTRACT

The present invention relates to a method for producing carbon nanomaterials and/or carbon micromaterials, in particular multi-wall carbon nanotubes. The method is characterized according to the invention in that at least one molecule that has a reactive group in terminal position is bound to the surface of the material. In addition, the invention also relates to a correspondingly modified material.

The present invention first relates to a method for producing carbon nanomaterials and/or carbon micromaterials, in particular multi-wall carbon nanotubes. In addition, the invention relates to a carbon nanomaterial and/or carbon micromaterial, in particular multi-wall carbon nanotubes.

In particular, the present invention relates to a covalent side-wall functionalization of multi-wall carbon nanotubes (CNTs).

For better wetting of CNTs and a possible covalent crosslinking of CNT surfaces to a reactive matrix surrounding them in order to improve mechanical properties, it is necessary to chemically modify the surfaces of CNTs.

Materials improved thereby based on crosslinking and thermoplastic plastics, are possible for in a wide range of applications, such as, for example, fiber composite components with improved interlaminar shearing strength, elastomers with elevated E-modulus, highly crosslinked resins with increased toughness, mechanically reinforced polyamides and the like.

In addition, improved dispersions based on aqueous or organic solvents can also be produced with surface-modified CNTs on the surface, and these dispersions then can be utilized as precursors for coatings, as additives in polymers, metals or ceramics.

In principle, CNT surfaces can be modified in different ways, which are generally known from the prior art, such as
Application of so-called surfactants, primarily tensides, which are bound to the surface of CNTs via Van der Waals interactions;
By coordinated polyaromatic compounds via a π-π interaction on the surface;
By growth of polymers on the surface of CNTs via “grafting from” methods;
By depositing metal or metal-oxide particles or films onto the CNT surface;
By oxidation of the CNT surface with oxidizing acids and further functionalization of the carboxyl groups that form:
By introducing molecules of low molecular weight with a specific terminal group via reactions that lead to a covalent binding to the CNT surface.

The last-named method is of particular interest for the application of CNTs as a reinforcing material in polymers, since here the strongest binding between the polymer matrix and the CNT-filler particles can be assured. Only by such a strong binding is it possible to obtain the reinforcing mechanisms known from composite material teaching, for example, force transfer to the embedded particles. In addition, the particular structure of CNTs is not too strongly attacked, as is the case with an oxidation of the surface, for which reason, both the mechanical properties as well as the electronic properties can be largely retained.

The thus-functionalized CNTs can be further derivatized and functionalized without problem via known methods and thus are suitable for a plurality of applications, for example, in resins. Thus, for example, covalent crosslinking of CNTs with polymer matrices and therefore an improvement in the mechanical and electronic properties of resins and of other polymers can be achieved.

The modification of CNTs with covalently bound molecules has been investigated for several years in different ways and has also already been utilized commercially for specific functionalities.

First, a distinction must be made between the functionalization of single-wall carbon nanotubes (SWCNTs) and multi-wall carbon nanotubes (MWCNTs). This is because there are fundamental differences in the type of reaction mechanisms that can be utilized at the present time for SWCNTs or MWCNTs, respectively.

A direct side-wall functionalization has currently only been demonstrated definitively for SWCNTs. This is explained by the stronger reactivity of SWCNTs based on the greater curvature of their surfaces and thus also by a greater pyramidalization of the π-system.

Oxidizing by means of previous methods, such as with HNO₃, for example, without seriously disrupting the properties of CNTs is only possible for MWCNTs. The oxidizing of SWCNTs by means of oxidizing acids or gases without too greatly damaging or completely destroying the SWCNTs is currently not possible.

At the present time, most studies on covalent functionalization of CNTs in the literature describe SWCNTs. The reasons for this are not generally known, but it is assumed that the great curvature of their surfaces and the strong pyramidalization of the π-system of SWCNTs resulting therefrom makes them particularly reactive, and thus first makes possible a chemical reaction of the it-bonds of the aromatic rings.

The reactions described for SWCNTs have previously not been definitively demonstrated for a direct side-wall functionalization of MWCNTs.

It was shown in more recent studies that the radius of curvature has an influence on the reaction, and the reactivity and thus the possibility of a covalent side-wall functionalization based on the radius of curvature would be very different for SWCNTs and MWCNTs. In particular, the radius of curvature for SWCNTs is clearly smaller* than for MWCNTs. Therefore, the previously described reactions, which had been demonstrated for SWCNTs, cannot be applied to MWCNTs under the given, maladaptive conditions, due to their clearly lower reactivity, which is attributable to the smaller radius of curvature of the external end wall of the tube.

For this reason, a multi-step pathway to functionalization has been previously usually carried out for MWCNTs. First, the surface of the MWCNTs is oxidized, for the most part, by means of concentrated nitric acid. In this process, of course, the π- and σ-bond system of the graphene layers is attacked and sensitively disrupted thereby, which has as a consequence a clear worsening of the mechanical properties and the chemical stability of the MWCNTs as well as a damaging of the same. This damaging also acts on layers found deeper inside, since this type of oxidizing, after disrupting the first side wall, also extends beyond it to the wall lying thereunder and also damages it. Thus, an uncontrolled disruption of the CNT on very defective regions of the CNT until the tube breaks is possible at this site.

At these oxidized sites, which are essentially composed of carboxyl groups, reactive centers are then created via another multi-step process by substitution of the carboxyl *sic; greater?—Translator's note groups with a halogen compound, for example, thionyl chloride, and more functional terminal groups can be introduced on these reactive centers.

The routes used for MWCNTs can often be carried out with strongly etching reagents, for example, thionyl chloride, and only with high cost for apparatus, for example, protective gas and necessary additional occupational safety measures due to the toxicity of the reagents.

Reaction routes that can directly functionalize the side wall without completely disrupting the side wall, and that can manage without highly toxic reagents and expensive apparatus are currently under investigation exclusively for SWCNTs.

Proceeding from the named prior art, the problem of the present invention is to provide a method for producing carbon nanomaterials and/or carbon micromaterials, in particular multi-wall carbon nanotubes, as well as a carbon nanomaterial and/or carbon micromaterial, in particular, multi-wall carbon nanotubes, in which the above-named disadvantages can be avoided.

The problem is solved according to the invention by the method with the features according to the independent patent claim 1, as well as the material with the features according to the independent patent claim 7. Further features and details of the invention can be taken from the subclaims, the description and the drawings. Features and details that are described in connection with the method thus also apply, of course, in connection with the material, and vice versa, so that relative to the disclosure of one aspect of the invention, reference is always made each time to the full extent to the disclosure of the other aspect of the invention.

In particular, the present invention makes possible a covalent—in particular, direct— side-wall functionalization of multi-wall carbon nanotubes.

According to the present invention, in particular, methods are described for producing molecules that are introduced covalently on the side wall of an MWCNT and that can be provided with selected functional terminal groups, without disrupting the side wall. In particular, the invention thus relates to a covalent side-wall functionalization of multi-wall carbon nanotubes (CNTs).

According to the first aspect of the invention a method is provided for producing carbon nanomaterials and/or carbon micromaterials, in particular multi-wall carbon nanotubes, which is characterized in that at least one molecule that has a reactive group in terminal position is bound to the surface of the material. This group, in particular, involves the above-named functional terminal group.

Carbon nanomaterials and carbon micromaterials, in particular, are microscopically small structures based on carbon, for example, composed of carbon. The size of carbon nanomaterials thus particularly lies in the nanometer range, while the size of carbon micromaterials particularly lies in the micrometer range. Of course, the present invention is generally not limited to specific carbon nanomaterials and/or carbon micromaterials. Preferably, however, the material involves multi-wall carbon nanotubes (MWCNTs). For this reason, reference is preferably made to this material in the further course of the description, but the invention is not limited to this one specific material.

Preferably the materials, in particular, the MWCNTs are provided with a molecule that is bound—especially covalently—to the surface, the molecule having a reactive group in terminal position, for example, a functional group or terminal group. “Reactive” therefore means capable of reaction, in particular. “Functional group” particularly involves a group that determines the material properties and/or the reaction behavior of compounds that bear it.

The covalent side-wall functionalization of MWCNTs, in particular with diamines, for producing end-terminal primary amino groups on the surface of the CNT side wall can be achieved by different pathways. Two preferred pathways are described in the following, the invention not being limited to the two named pathways.

Advantageously, the binding can be achieved by means of a diazotizing reaction and/or a condensation reaction. Here, the molecule is bound to the CNT surface via a diazotizing reaction and/or a condensation reaction. In this case, the functionalization is achieved, for example, by a diazotizing of the side wall adjusted to the difficult conditions of reactions with MWCNTs (pathway I) and/or by a fluorination of the CNT sidewall and subsequent condensation with a diamine (pathway II).

The pathway of diazotizing by means of a diazotizing reaction (pathway I) will be described in greater detail in the following by way of example.

Preferably, for the diazotizing, MWCNTs that are particularly purified are provided dry or as a dispersion, for example, with an amine and sodium nitrite or isoamyl nitrite. Water or different organic solvents such as methanol, ethanol, butanol, toluene, DMF, THF, or the like, can be used as the solvent for the dispersions. For better dispersing, a dispersing agent is advantageously added to the dispersion, for example, any tenside-acting dispersing agent with an anionic, non-ionic or cationic character, for example, Triton X 100, SDS, PEI or similar agent. The dispersing agent is selected depending on the type of solvent used, relative to both its type and concentration in each case. The concentration of CNTs in the solvent can advantageously amount to between 0.001 and 10 weight percent. Only a sufficiently good segregating of the CNTs in the dispersion with a simultaneous lower viscosity of the dispersion, for example 10 mPa·s to 1 Pa·s, is important for facilitating a stirring of the dispersion.

After this, the reaction mixture can be flushed with protective gas, for example, CO₂, N₂, Ar, He, Ne, and reacted with a mineral acid, for example HCl, H₂SO₄, HNO₃, H₃PO₄ at a suitable temperature, for example between 0° C. and 50° C., under vigorous stirring. The described diazotizing is preferably conducted with the above-named protective gases, but can be successfully carried out even without preliminary removal of the oxygen. The mineral acid must be present at the selected temperature in the liquid aggregate state. The concentration of the mineral acid is advantageously selected so that the pH value of the reaction solution lies between pH=1 and pH=6.5.

The reaction mixture is subsequently stirred advantageously for between 10 minutes and 600 minutes and can then be cooled to a suitable temperature, for example between 0° C. and 30° C.

The reaction product is advantageously washed with a large amount of pure solvent, such as, for example, methanol, ethanol, butanol, toluene, DMF, THF, and/or water and subsequently dried.

An example of embodiment for the diazotizing will be described in the following.

According to this example, purified MWCNTs are provided dry and dispersed in p-phenylendiamine and sodium nitrite for the diazotizing. DMF is used as a solvent for the dispersions and 0.5 weight percent of Triton X 100 is added as a dispersing agent. The concentration of CNTs in the solvent amounts to 0.01 weight percent. Then the reaction mixture is flushed with CO₂ and reacted with concentrated HCl at 10° C. with vigorous stirring. The mineral acid must be present at the selected temperature in the liquid aggregate state. The reaction mixture is subsequently stirred for 30 min at 50° C. and cooled to room temperature (RT). The reaction product is washed with a large amount of ethanol and dried.

A selection of different possible reaction partners for this pathway I is given in the following Table 1.

| | |
|---|---|
| p-Phenylendiamine | 4-Nitroaniline |
| 4-Chloraniline | 4-Bromaniline |
| Adipic acid dihydrazide | 4-Aminobenzamide |
| 4-Aminoazobenzene | 4-Aminobenzonitrile |
| 4-Aminopyridine | Benzylamine |
| 4-Aminobenzoic acid | 4-Hydroxyaniline |

The pathway for a condensation by means of a condensation reaction (pathway II) is described in greater detail below by way of example.

For the condensation reaction, MWCNTs that are advantageously halogenated, for example with F, CI, Br or I, are dispersed with different, preferably aliphatic, diamines, preferably having a carbon chain length between 2 and 10 carbon atoms, for example, in water or organic solvents, for example methanol, ethanol, butanol, toluene, DMF, THF, and preferably flushed with protective gas, for example CO₂, N₂, Ar, He, Ne, preferably with ultrasound treatment at suitable temperatures, advantageously between 20° C. and 120° C. After this, the reaction mixture is preferably heated to a suitable temperature, for example between 70° C. and 170° C. After reaching the reaction temperature, pyridine is advantageously added and stirring is conducted over a suitably long period of time, for example, between 1 h and 10 h. After this, the reaction product is advantageously cooled to a suitable temperature, for example between 0° C. and 30° C., and advantageously washed with a large amount of solvent.

An example of embodiment for the condensation will be described in the following.

According to this example, purified dry MWCNTs are provided along with 1,6-diaminohexane in toluene. The mixture is first dispersed by means of a rapid-rotating high-power mixing device and then flushed thoroughly with helium in an ultrasound bath. After this, the dispersion is heated to 130° C. The pyridine is added to this and the mixture is kept for 360 minutes at this temperature with constant stirring. After this reaction time, the dispersion is cooled to 30° C. and filtered, and washed with a large amount of butanol. Subsequently, the powder is dried.

The condensation can be conducted both with the above-named substances as well as their derivatives, and diamino alkanes, alkenes and alkynes with a C chain length of 2 to 30 C atoms. Here, aromatic reaction partners such as p-phenylenediamine, alkenes and alkynes lead to rather more rigid functional groups, whereby the binding to polymer systems is further facilitated, since steric hindrance is minimized.

A selection of different possible reaction partners for this pathway II is given in the following Table 2.

| | |
|---|---|
| 1,4-Diaminobutane | 1,5-Diaminopentane |
| 1,6-Diaminohexane | 1,10-Diaminodecane |
| 1,4-Diaminobut-2-ene | 1,4-Diaminobut-2-yne |

Both described reactions according to pathway I and pathway II must be adapted to the special reaction conditions with MWCNTs. As already described further above, SWCNTs and MWCNTs are not trivially comparable with one another due to their different degrees of pyramidalization of then-system, which means that the methods developed for SWCNTs on the milligram scale are not transferable to MWCNTs and hardly to reactions with MWCNTs on a technical scale.

Since the previous methods were always directed to the use of toxic or hazardous solvents such as dichlorobenzene or THF, these are additionally unsuitable for application on a technical and mass-production scale for producing functionalized CNTs on the scale of tons. Thus, a non-hazardous reaction pathway that is as simple as possible, as well as an environmentally-friendly disposal of chemical wastes and solvents, and an upscaling that is as simple as possible, were respected in the development of these methods.

The reactions were carried out successfully for both pathways on scales from 50 mg up to 10 grams and offer a simple possibility for the covalent side-wall functionalization of MWCNTs with diamines and their derivatives for the binding of end-terminal functional amino groups. Both methods open up a broad range of additional functionalizations and have a high variability of functional groups and thus a broad spectrum of different applications in the field of composites.

Both processes are advantageously one-stage processes and thus, in combination with the purification, lead to the desired reaction product in only two steps. Based on these few operating steps, the processes are superior to conventional methods, which, after the purification, require a carboxylating step, an acid activation with thionyl chloride or similar substances, followed by an esterification or amination, respectively, of the acid group and the necessary purification.

Therefore, the processes presented here, when compared with conventional methods, are more cost-effective, less labor-intensive and, in particular, more environmentally friendly.

In addition, the developed methods, in particular, offer a direct linking of the functional group to the side wall of the MWCNT without damaging the a-bond structure. Due to the bond formation, in particular, the sp²-hybridized carbon atoms are converted to an sp³-hybridization, or the existing sp³-hybridized carbon atoms produced by the fluorination are aminated under HF condensation. Therefore, neither of the two methods presented is dependent on defects in the tube structure or on a dissolution of the σ-bond structure of the MWCNTS, as is the case with conventionally used carboxylation.

According to an advantageous enhancement of the method, the molecule can thus be bound to carbon nanotubes, in particular, to purified carbon nanotubes and/or to pre-functionalized carbon nanotubes. In particular, the reactions can be carried out on purified CNTs and pre-functionalized CNTs, for example, fluorinated CNTs.

Preferably, the method is designed in such a way that all educts named in the Claims, the Description, the Examples, the Tables and the Drawings can be utilized as molecules and/or as a functional group.

Advantageously, the method can be conducted under conditions that exclude the use of organic solvents. A particularly environmentally friendly and very simple method from the point of view of occupational health and safety and method technology is made possible in this way.

Preferably, the method can be carried out in batch operation. Alternatively, however, the method may also be conducted in continuous operation.

The method is advantageously designed in such a way that it has one or more of the features named in the Claims, the Description, the Examples, the Tables and the Drawings.

Preferably, the binding of the functional group or group or reactive group can be achieved without damage or disruption of the a-bond system. Thus, the present invention makes possible a covalent binding without disruption of the σ-bond system, in particular for both of the described pathways I and II.

According to the second aspect of the invention a carbon nanomaterial and/or carbon micromaterial, in particular multi-wall carbon nanotubes, is provided, which is characterized in that at least one molecule that has a reactive group in terminal position is bound to the surface of the material.

It is thus advantageously provided that the material is produced, has been produced, or can be produced by means of a method according to the invention, as described above, so that reference is made to the full extent to the statements relative to the method according to the invention.

Preferably, the material is configured in a way that it has one or more of the features named in the Claims, the Description, the Examples, the Tables and the Drawings.

The invention will now be explained in more detail based on the embodiment examples with reference to the attached drawing. Here:

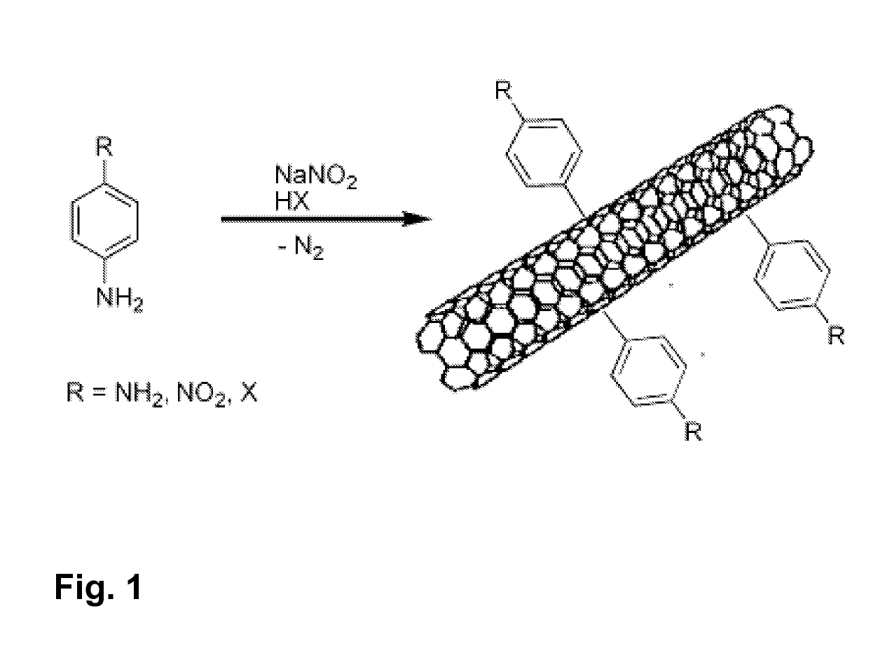

FIG. 1 shows the reaction pathway of a covalent functionalization according to pathway I; and

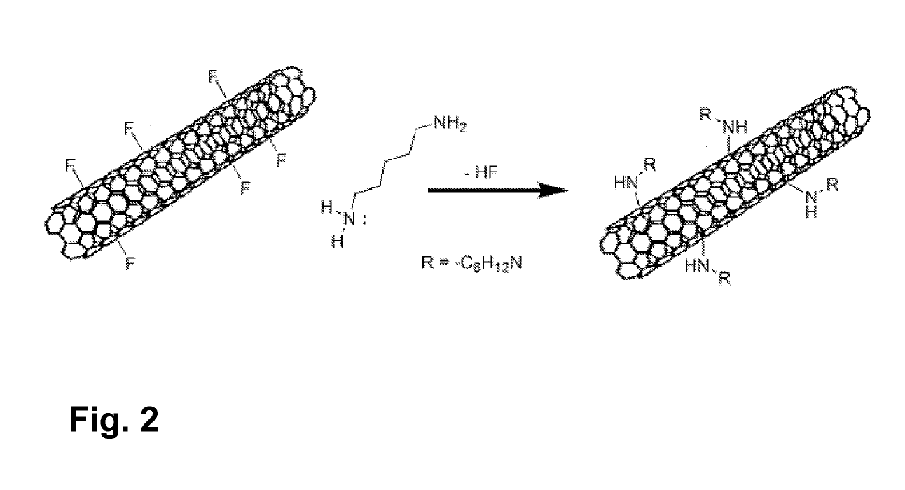

FIG. 2 shows the reaction pathway of a covalent functionalization according to pathway II.

The products that form by means of the methods described further above are shown schematically in FIGS. 1 and 2, in order to further clarify the type of bond and the different possibilities. The above-named Tables 1 and 2 list different diamines, such as the most varied aniline derivatives, hydrazines, diamino alkanes, alkenes and alkynes, as well as azo dyes with functional amino groups, aminobenzene and aminopyridine derivatives, which are considered as reaction partners.

FIG. 1 shows, in particular, the reaction pathway of the covalent amino functionalization via pathway I; with X=Cl, Br. FIG. 2 shows, in particular, the reaction pathway of the covalent amino functionalization via pathway II.

1. A method for producing carbon nanomaterials and/or carbon micromaterials, in particular multi-wall carbon nanotubes, is hereby characterized in that at least one molecule that has a reactive group in terminal position is bound to the surface of the material.

2. The method according to claim 1, further characterized in that the binding is achieved by means of a diazotizing reaction and/or a condensation reaction.

3. The method according to claim 1, further characterized in that the molecule is bound to carbon nanotubes, in particular, to purified carbon nanotubes and/or to pre-functionalized carbon nanotubes.

4. The method according to claim 1, further characterized in that it is conducted under conditions that exclude the use of organic solvents:

5. The method according to claim 1, further characterized in that it is carried out in batch operation or that it is carried out in continuous operation.

6. The method according to claim 1, further characterized in that the binding of the functional group is achieved without damage or disruption of the σ-bond system.

7. A carbon nanomaterial and/or carbon micromaterial, in particular multi-wall carbon nanotubes, is hereby characterized in that at least one molecule that has a reactive group in terminal position is bound to the surface of the material.

8. A carbon nanomaterial and/or carbon micromaterial, in particular multi-wall carbon nanotubes, is hereby characterized in that at least one molecule that has a reactive group in terminal position is bound to the surface of the material and in that it is produced, has been produced or can be produced with a method according to claim 1.

9. The carbon nanomaterial and/or carbon micromaterial as claimed in claim 8, further characterized in that the binding is achieved by means of a diazotizing reaction and/or a condensation reaction.

10. The carbon nanomaterial and/or carbon micromaterial as claimed in claim 8, further characterized in that the molecule is bound to carbon nanotubes, in particular, to purified carbon nanotubes and/or to pre-functionalized carbon nanotubes.

11. The carbon nanomaterial and/or carbon micromaterial as claimed in claim 8, further characterized in that it is conducted under conditions that exclude the use of organic solvents.

12. The carbon nanomaterial and/or carbon micromaterial as claimed in claim 8, further characterized in that it is carried out in batch operation or that it is carried out in continuous operation.

13. The carbon nanomaterial and/or carbon micromaterial as claimed in claim 8, further characterized in that the binding of the functional group is achieved without damage or disruption of the σ-bond system.
